(19) United States
(12) Patent Application Publication
(10) Pub. No.: US 2007/0183995 A1
(43) Pub. Date: Aug. 9, 2007

(54) Compounds useful as agonists of A2A adenosine receptors, cosmetic compositions with A2A agonists and a method for using the same

(75) Inventors: John Chun-Sing Nip, Shelton, CT (US); Carol Annette Bosko, Newtown, CT (US); Jose Guillermo Rosa, Shelton, CT (US); Bijan Harichian, Brookfield, CT (US); Isabel Cristina Santana, Harrington Park, NJ (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a Unilever

(21) Appl. No.: 11/350,658

(22) Filed: Feb. 9, 2006

(51) Int. Cl. A61K 8/49; C07H 19/16

(52) U.S. Cl. 424/62; 536/27.3

(57) ABSTRACT

Compounds useful as agonists of A_{2A} adenosine receptors are described. Also described is a cosmetically acceptable composition having an agonists of A_{2A} adenosine receptors where the composition is suitable to apply to human skin to reduce the effects of melanin, resulting in skin whitening.

### FIELD OF THE INVENTION

The present invention is directed to compounds useful as agonists of A_{2A} adenosine receptors. The present invention is also directed to a cosmetic composition, and a method for improving skin characteristics by using the same. More particularly, the invention is directed to a cosmetic composition comprising, as an active agent, an agonist of A_{2A} adenosine receptors. The composition of the present invention, at the very least and surprisingly, lightens skin, evident by the fact that a ΔL of at least about 0.3 is measured when the same is applied to melanoderm cultures and compared to control melanoderm cultures that have not been subjected to the composition.

### BACKGROUND OF THE INVENTION

Many consumers are concerned with the characteristics of their skin. For example, consumers are concerned with the degree of pigmentation of their skin, freckles and/or age spots. Moreover, consumers also seek to alleviate or delay the signs of aged or photo-aged skin as well as dry and sagging skin. Others wish to reduce skin darkening caused by exposure to sunlight. To meet the needs of consumers, many attempts have been made to develop products that improve skin characteristics. The products developed thus far, however, tend to have low efficacy or undesirable side effects, such as, for example, toxicity or skin irritation.

There is an increasing interest to develop a cosmetic composition that lightens skin in the absence of side effects. This invention, therefore, is directed to a side-effect free cosmetic composition that, at the very least and surprisingly, lightens skin. The cosmetic composition of the present invention comprises, as an active agent, an agonist of A_{2A} adenosine receptors, and results in a ΔL of at least about 0.3 when comparing melanoderm cultures treated with the same to melanoderm cultures that have not been subjected to a composition with agonists of A_{2A} adenosine receptors.

### ADDITIONAL INFORMATION

Efforts have been disclosed for making skin care cosmetic compositions. In U.S. Pat. No. 6,875,425, skin lightening agents with 4-substituted resorcinol derivative compounds are described.

Other efforts have been disclosed for making skin treatment compositions. In U.S. Application Publication No. 2004/0071749 A1, methods for treating skin with adenosine or adenosine analogs are described.

Still other efforts have been disclosed for treating skin. In U.S. Pat. No. 5,998,423, compositions with polycyclic nitrogen heterocycles are described.

None of the additional information above describes compounds that are agonist of A_{2A} adenosine receptors wherein the same are suitable for use in a composition that results in skin lightening.

### SUMMARY OF THE INVENTION

In a first aspect, the present invention is directed to compounds comprising the formula: 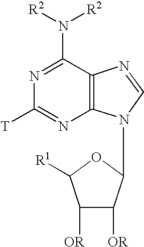
wherein
(a) each R is independently hydrogen, a C₁-C₂₀ linear, branched, substituted, unsubstituted, saturated and/or unsaturated alkyl, acyl group or aryl group;
(b) R¹ is a C₁-C₅ alkanol or 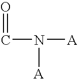
where each A is independently hydrogen or a C₁-C₅ alkyl; and
(c) T is a group comprising at least one heteroatom with the provisos that T has a heteroatom selected from the group consisting of N, O and S bonded to purine and when T is 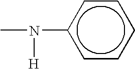
each R and R² are not simultaneously H when R¹ is CH₂OH, and when T is 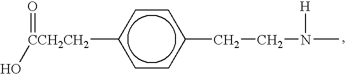
each R and R² are not simultaneously hydrogen when R¹ is

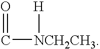

In a second aspect, the invention is directed to a cosmetic composition suitable to at least lighten skin and comprising an agonist of A_{2A} adenosine receptors.

In a third aspect, the invention is directed to a method for lightening skin.

As used herein, ΔL is defined to mean the difference in Hunter Lab L values when comparing three, three (3) week old Mattek Melanoderm cultures that have not been treated with a composition comprising an agonist of A_{2A} receptors to three, three (3) week old Mattek melanoderm cultures that have been treated with a composition comprising an agonist of A_{2A} receptors wherein treated means:
(a) placing the melanoderm culture within a six (6) well tissue culture dish and set about 0.3 cm off of the tissue culture dish;
(b) subjecting the melanoderm culture to a 3 micromolar composition having an agonist of A_{2A} adenosine receptors, the composition being one prepared from a 10 millimolar solution of A_{2A} agonist and carrier (e.g., dimethyl sulfoxide) having been diluted with Dulbecco's Modified Eagle Media; and
(c) comparing the treated and untreated cultures by obtaining average L values for each with a Minolta CR-10 chromameter.

Cosmetic composition is meant to include a composition for topical application to skin of mammals, especially humans. Such a composition may be generally classified as leave-on or rinse off, and is meant to include conditioners or tonics, lipsticks, color cosmetics, and general topical compositions that in some fashion and at the very least, reduce the effect of melanin on keratinocytes. Lightening and whitening as used herein are meant to mean the same and they include the lightening of skin directly as well as the lightening of spots on the skin, like age spots and freckles. Dulbecco's Modified Eagle Media means the nutrient solution sold by Mattek and treated and used according to instructions supplied with the product commercially identified as MEL30010BBLLMM.

The composition of the present invention can be in the form of a liquid, lotion, cream, gel, soap bar or toner, or applied via a face mask or patch. The composition of this invention is one that at the very least, lightens skin when skin is meant to include skin on the face, neck, chest, back, arms, hands, legs and scalp. All ranges identified herein are meant to implicitly include all ranges subsumed therein if, for example, reference to the same is not explicitly made.

### DETAILED DESCRIPTION OF THE PREFERRED EMBODIMENTS

In one embodiment, the present invention is directed to compounds comprising the formula: 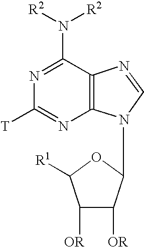
wherein
(a) each R is independently hydrogen, a C₁-C₂₀ linear, branched, substituted, unsubstituted saturated and/or unsaturated alkyl, acyl group or aryl group;
(b) R¹ is a C₁-C₅ alkanol or 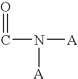
where each A is independently hydrogen or a C₁-C₅ alkyl; and
(c) T is a group comprising at least on heteroatom with the provisos that T has a heteroatom selected from the group consisting of N, O and S bonded to purine and when T is 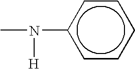
each R and R² are not simultaneously H when R¹ is CH₂OH, and when T is 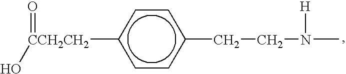
each R and R² are not simultaneously hydrogen when R¹ is

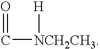

In an often preferred embodiment, T is 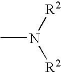
where each R² is independently
(a) hydrogen, a C₁-C₂₀ linear, branched, cyclic, saturated or unsaturated alkyl group with or without a heteroatom selected from the group consisting of N, O and S, an aryl group, alkyl aryl, C₄-C₉ heteroaryl, C₄-C₁₀ heterocycle where the heteroatom is selected from the group consisting of N, O and S, 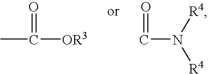
where R³ is a C₁-C₂₀ linear, branched, saturated or unsaturated alkyl group with or without a heteroatom selected from the group consisting of N, O and S, and each R⁴ is independently hydrogen, C₁-C₂₀ linear, branched, saturated or unsaturated alkyl group with or without a heteroatom selected from the group consisting of N, O and S, with the provisos that when T is 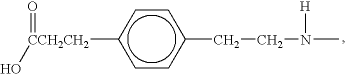
   each R and R² are not simultaneously hydrogen when R¹ is

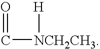

Regarding the cosmetic composition of this invention, any agonists of A_{2A} adenosine receptors may be employed as long as they are suitable for use with skin, especially human skin, and able to reduce the effect of melanin on kerotinocytes. In a preferred embodiment, the agonist of A_{2A} adenosine receptors is adenosine derived and free of a carbon-carbon triple bond directly bonded to the purine portion of the adenosine derived agonist. In another preferred embodiment, the agonist of A_{2A} adenosine receptors suitable for use in this invention is represented by the formula above with the exception that phenylaminoadenosine and 2-para (2-carboxyethyl)phenethylamino-5′-N-ethyl carboxamido adenosine may be used, and most preferably, are used either alone or in combination with each other.

When formulating the cosmetic composition of the present invention, the agonist of A_{2A} adenosine receptors is present in an amount effective to lighten skin. Typically, such an agonist results in a ΔL of at least about 0.3 as defined herein, and preferably, from about 0.75 to about 6.5, and most preferably, from about 1.0 to about 5.5, including all ranges subsumed therein. Typically, the amount of agonist used in the cosmetic composition is from about 0.0001 to about 10%, and preferably, from about 0.01 to about 5%, and most preferably, from about 0.1 to about 1% by weight based on total weight of the cosmetic composition and including all ranges subsumed therein.

It should be known that commercially acceptable and conventional vehicles may be used, acting as diluents, dispersants and/or carriers for the agonists described herein and for any other optional but often preferred additives. Therefore, the cosmetically acceptable vehicle suitable for use in this invention may be aqueous-based, anhydrous or an emulsion whereby a water-in-oil or oil-in-water emulsion is generally preferred. If the use of water is desired, water typically makes up the balance of the cosmetic composition, and preferably, makes up from about 5 to about 99%, and most preferably, from about 40 to about 80% by weight of the cosmetic composition, including all ranges subsumed therein.

In addition to water, organic solvents may be optionally included to act as carriers or to assist carriers within the compositions of the present invention. Illustrative and non-limiting examples of the types of organic solvents suitable for use in the present invention include alkanols like methyl, ethyl and isopropyl alcohol, mixtures thereof or the like.

Other optional additives suitable for use include ester oils like isopropyl myristate, cetyl myristate, 2-octyldodecyl myristate, avocado oil, almond oil, olive oil, neopentylglycol dicaprate, mixtures thereof or the like. Typically, such ester oils assist in emulsifying the cosmetic composition of this invention, and an effective amount is often used to yield a stable, and most preferably, water-in-oil emulsion.

Emollients may also be used, if desired, as carriers within the cosmetic composition of the present invention. Alcohols like 1-hexadecanol (i.e., cetyl alcohol) and phenoxyethanol are often desired as are the emollients generally classified as silicone oils and synthetic esters. Silicone oils suitable for use include cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material in the inventive cosmetic composition described herein include polyalkyl siloxanes, polyalkylaryl siloxanes and polyether siloxane copolymers. The essentially non-volatile polyalkyl siloxanes useful herein include, for example, polydimethylsiloxanes with viscosities of from about 5 to about 25 million centistokes at 25° C. Among the preferred non-volatile emollients useful in the present compositions are the polydimethylsiloxanes having viscosities from about 10 to about 400 centistokes at 25° C.

The ester emollients that may optionally be used are:
(1) Alkenyl or alkyl esters of fatty acids having 10 to 20 carbon atoms. Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono-and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, stearyl stearate and arachidyl behenate.
(5) Sterols esters, of which cholesterol fatty acid esters are examples.

Emollients when used, typically make up from about 0.1 to about 50% by weight of the cosmetic composition, including all ranges subsumed therein.

Fatty acids having from 10 to 30 carbon atoms may also be included as cosmetically acceptable carriers within the composition of the present invention. Illustrative examples of such fatty acids include pelargonic, lauric, myristic, palmitic, stearic, isostearic, hydroxystearic, oleic, linoleic, ricinoleic, arachidic, behenic or erucic acid, and mixtures thereof. Compounds that are believed to enhance skin penetration, like dimethyl sulfoxide, may also be used as an optional carrier.

Humectants of the polyhydric alcohol type may also be employed in the cosmetic compositions of this invention. The humectant often aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.2 to 25%, and preferably, from about 0.5 to about 15% by weight of the cosmetic composition, based on total weight of the cosmetic composition and including all ranges subsumed therein.

Thickeners may also be utilized as part of the cosmetically acceptable carrier in the cosmetic compositions of the present invention. Typical thickeners include cross-linked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0 to 5%, usually from 0.001 to 1%, optimally from 0.01 to 0.5% by weight.

Collectively, the water, solvents, silicones, esters, fatty acids, humectants and/or thickeners will constitute the cosmetically acceptable carrier in amounts from 1 to 99.9%, preferably from 80 to 99% by weight.

Surfactants may also be present in cosmetic compositions of the present invention. Total concentration of the surfactant will range from about 0 to about 40%, and preferably, from about 0 to about 20%, optimally from about 1 to about 5% by weight of the composition. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di- fatty acid esters of ethylene glycol; fatty acid monoglyceride; sorbitan, mono- and di- C₈-C₂₀ fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isothionates, acyl glutamates, C₈-C₂₀ alkyl ether phosphates and combinations thereof.

Perfumes may be used in the cosmetic composition of this invention. Illustrative non-limiting examples of the types of perfumes that may be used include those comprising terpenes and terpene derivatives like those described in Bauer, K., et al., Common Fragrance and Flavor Materials, VCH Publishers (1990)).

Illustrative yet non-limiting examples of the types of fragrances that may be used in this invention include myrcene, dihydromyrenol, citral, tagetone, cis-geranic acid, citronellic acid, or cis-geranic acid nitrile, mixtures thereof or the like.

Preferably, the amount of fragrance employed in the cosmetic composition of this invention is in the range from about 0.0% to about 10%, more preferably, about 0.00001% to about 5 wt %, most preferably, about 0.0001% to about 2%.

Various types of optional additional active ingredients may be used in the cosmetic compositions of the present invention. Actives are defined as skin benefit agents other than emollients and other than ingredients that merely improve the physical characteristics of the composition. Although not limited to this category, general examples include talcs and silicas, as well as alpha-hydroxy acids, beta-hydroxy acids, poly-hydroxy acids, peroxides, zinc salts, and sunscreens.

Beta-hydroxy acids include salicylic acid, for example. Zinc pyrithione is an example of the zinc salts useful in the cosmetic composition of the present invention.

Sunscreens include those materials commonly employed to block ultraviolet light. Illustrative compounds are the derivatives of PABA, cinnamate and salicylate. For example, avobenzophenone (Parsol 1789®) octyl methoxycinnamate and 2-hydroxy-4-methoxyl benzophenone (also known as oxybenzone) can be used. Octyl methoxycinnamate and 2-hydroxy-4-methoxy benzophenone are commercially available under the trademarks, Parsol MCX and Benzophenone-e, respectively. The exact amount of sunscreen employed in the compositions can vary depending upon the degree of protection desired from the sun's UV radiation. Additives that reflect or scatter the suns rays may also be employed. These additives include oxides like zinc oxide and titanium dioxide.

Many cosmetic compositions, especially those containing water, should be protected against the growth of potentially harmful microorganisms. Anti-microbial compounds, such as triclosan, and preservatives are, therefore, typically necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

Still other optional ingredients that may be used with the cosmetic composition of this invention include dioic acids (e.g., malonic acid, sebacic acid), vitamins, like niacinamide, vitamin C, recorcinols and its derivatives (including those esterified with, for example, ferulic acid, vanilla acid or the like) and retinoids, including retinoic acid, retinal, retinal and retinyl esters, as well as any other conventional ingredients well known for wrinkle-reducing, skin whitening, anti-acne effects and reducing the impact of serum.

The cosmetic compositions of the present invention are intended for use primarily as a product for topical application to human skin, especially and at least as an agent for lightening the skin. Other benefits may include skin moisturizing, decreasing the effect of serum on the skin and skin wrinkle reducing. Often, the cosmetic composition of the present invention has a melting point from about 30° C. to about 45° C., including all ranges subsumed therein.

When making the cosmetic composition of the present invention, the desired ingredients are mixed in no particular order and usually at temperatures from about 70 to about 80° C. and under atmospheric pressure. The agonists described herein are made via methods which can include esterification reactions and/or reductions.

The packaging for the composition of this invention can be a patch, bottle, tube, roll-ball applicator, propellant driven aerosol device, squeeze container or lidded jar.

The example below is provided to illustrate the invention and are not intended to limit the scope of the claims.

### EXAMPLE

Commercially available human skin equivalents (Melanoderm from Mattek) were obtained for testing the impact of agonists of A_{2A} adenosine receptors on melanogenesis. Solutions having a final concentration of three (3) micromolar were prepared from a 10 millimolar dimethyl sulfoxide stock solution and dosed ten (10) times in a three (3) week period into the media of the melanoderm cultures. The media consisted of commercially available basal Dulbecco's Modified Eagles media, prepared and treated in the manner set forth in the manufacture's instructions. For long term maintenance of the Melanoderms, the basal media was supplemented with bFGF and alpha MSH to stimulate melanocyte growth and melanogenesis. Each treatment condition was done in triplicate and three (3) sets were made for each treatment, as well as for a control (culture not treated with the agonist). The cultures were maintained at a temperature of about 37° C. and stored in a humidified, 5% CO₂ incubator during the dosing period, but removed while being dosed.

After a three (3) week period, Hunter lab L value readings were taken for each condition (with a.Minolta CR-10 chromameter) and then averaged. The results are provided below:

TABLES 1

| Active | L Value Range | Average L Value | ΔL |
|---|---|---|---|
| Control | 29.9-30.8 | 38.6 | — |
| Agonist 1ⁱ | 30.3-33.3 | 31.9 | 1.7 |
| Control | 38.2-39.3 | 38.6 | — |
| Agonist 2ⁱⁱ | 43.2-44.2 | 43.8 | 5.2 |

ⁱ= 2-para(2-carboxyethyl)phenethylamino-5′-N-ethyl carboxamido adenosine
ⁱⁱ= phenylaminoadenosine

The results, as they relate to melanoderm cultures, show that compositions with an agonist of A_{2A} adenosine receptors can unexpectedly result in skin lightening.

What is claimed is:

1. A compound comprising the formula 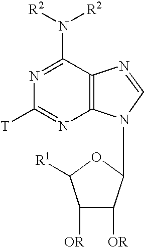
wherein
(d) each R is independently hydrogen, a C₁-C₂₀ linear, branched, substituted, unsubstituted saturated and/or unsaturated alkyl, acyl group or aryl group;
(e) R¹ is a C₁-C₅ alkanol or 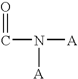
where each A is independently hydrogen or a C₁-C₅ alkyl; and
(f) T is a group comprising at least on heteroatom with the provisos that T has a heteroatom selected from the group consisting of N, O and S bonded to purine and when T is 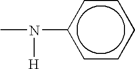
each R and R² are not simultaneously H when R¹ is CH₂OH, and when T is 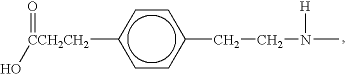
each R and R² are not simultaneously hydrogen when R¹ is

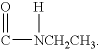

2. A method for lightening skin comprising the step of contacting the skin with a composition comprising an effective amount of an agonist of A_{2A} adenosine receptors, the effective amount being enough to lighten skin.

3. The method for lightening skin according to claim 2 wherein the agonist of A_{2A} adenosine receptors is present in the composition in an amount from about 0.0001 to about 10% by weight.

4. The method for lightening skin according to claim 2 wherein the agonist of A_{2A} adenosine receptors is present in the composition in an amount from about 0.01 to about 5% by weight.

5. The method for lightening skin according to claim 2 wherein the agonist of A_{2A} adenosine receptors is present in the composition in an amount from about 0.1 to about 1% by weight.

6. The method for lightening skin according to claim 2 wherein the agonist of A_{2A} adenosine receptors is one which results in a ΔL of at least about 0.3 when comparing three, three (3) week old Mattek Melanoderm cultures that have not been treated with the composition comprising the agonist of A_{2A} receptors to three, three (3) week old Mattek melanoderm cultures that have been treated with the composition comprising the agonist of A_{2A} receptors wherein treated means:
(a) placing the melanoderm culture within a six (6) well tissue culture dish and set about 0.3 cm off of the tissue culture dish;
(b) subjecting the melanoderm culture to a 3 micromolar composition having an agonist of A_{2A} adenosine receptors, the composition being one prepared from a 10 millimolar solution of A_{2A} agonist and carrier (e.g., dimethyl sulfoxide) having been diluted with Dulbecco's Modified Eagle Media; and
(c) comparing the treated and untreated cultures by obtaining average L values for each with a Minolta CR-10 chromameter.

7. The method for lightening skin according to claim 6 wherein the agonist of A_{2A} adenosine receptors is one which results in a ΔL from about 0.75 to about 6.5.

8. The method for lightening skin according to claim 6 wherein the agonist of A_{2A} adenosine receptors is one which results in a ΔL from about 1.0 to about 5.5.

9. The method for lightening skin according to claim 2 wherein the agonist of A_{2A} adenosine receptors is a compound as represented in claim 1.

10. The method for lightening skin according to claim 2 wherein the agonist of A_{2A} adenosine receptors is phenylaminoadenosine, 2-para (2-carboxyethyl)phenethylamino-5′-N-ethyl carboxamido adenosine or a mixture thereof.

11. The method for lightening skin according to claim 2 wherein the composition further comprises additional active ingredients selected from talcs, silicas, alpha hydroxy acids, beta-hydroxy acids, poly-hydroxy acids, peroxides, zinc salts, sunscreens, dioic acid or vitamin.

12. The method for lightening skin according to claim 2 wherein the composition further comprises an additional active ingredient classified as a wrinkle-reducing agent, skin whitening agent, anti-acne agent, an agent to reduce the impact of serum or a mixture thereof.

13. A composition comprising:
(a) an agonist of A_{2A} adenosine receptors;
(b) a cosmetically acceptable carrier
wherein the agonist of A_{2A} adenosine receptors is one which results in a ΔL of at least about 0.3 when comparing three, three (3) week old Mattek Melanoderm cultures that have not been treated with the composition comprising the agonist of A_{2A} receptors to three, three (3) week old Mattek melanoderm cultures that have been treated with the composition comprising the agonist of A_{2A} receptors wherein treated means:
(a) placing the melanoderm culture within a six (6) well tissue culture dish and set about 0.3 cm off of the tissue culture dish;
(b) subjecting the melanoderm culture to a 3 micromolar composition having an agonist of A_{2A} adenosine receptors, the composition being one prepared from a 10 millimolar solution of A_{2A} agonist and carrier (e.g., dimethyl sulfoxide) having been diluted with Dulbecco's Modified Eagle Media; and
(c) comparing the treated and untreated cultures by obtaining average L values for each with a Minolta CR-10 chromameter.

14. The composition according to claim 13 wherein the agonist of A_{2A} adenosine receptors is one which results in a ΔL from about 0.75 to about 6.5.

15. The composition according to claim 13 wherein the agonist of A_{2A} adenosine receptors is one which results in a ΔL from about 1.0 to about 5.5.

16. The composition according to claim 13 wherein the agonist of A_{2A} adenosine receptors is a compound as represented in claim 1.

17. The composition according to claim 13 wherein the agonist of A_{2A} adenosine receptors is phenylaminoadenosine, 2-para (2-carboxyethyl)phenethylamino-5′-N-ethyl carboxamido adenosine or a mixture thereof.
